(19) United States
(12) Patent Application Publication
(10) Pub. No.: US 2011/0015597 A1
(43) Pub. Date: Jan. 20, 2011

(54) Multiple Component Materials Having A Color-Changing Composition

(76) Inventors: JunMo Gil, TeaJeon (KR); SeungRim Yang, Seongnam-si (KR); Xuedong Song, Alpharetta, GA (US)

Correspondence Address: KIMBERLY-CLARK WORLDWIDE, INC.;Tara Pohlkotte, 2300 Winchester Rd., NEENAH, WI, 54956, US

(21) Appl. No.: 12/503,364

(22) Filed: Jul. 15, 2009

(51) Int. Cl. A61F 13/42; B32B 5/16; B32B 27/12; B32B 27/32; B32B 27/34; B32B 27/40; B32B 3/10; B05D 3/02

(52) U.S. Cl. 604/361; 428/327; 442/394; 428/523; 428/476.9; 428/424.8; 428/195.1; 427/385.5

(57) ABSTRACT

The present invention relates to a multiple-component material including a substrate and a film layer on the substrate. The film layer includes a color-changing composition to indicate a change in condition, such as a change in pH. The color-changing composition includes a water-insoluble, film-forming polymer, a pH adjuster and a water-insoluble particle. The water-insoluble particle has a charged colorant immobilized on it.

### FIELD OF THE INVENTION

The present invention relates to multiple-component materials that include a substrate and a film layer formed on the substrate. The film layer includes a color-changing composition. The color-changing composition can cause the film layer to change in appearance of its color when the film layer is exposed to a change in physical or chemical environment. For example, the film layer may be on a nonwoven material that is used as a component of an absorbent article. The color-changing composition may include a pH indicator that changes color in response to the presence of urine.

### BACKGROUND OF THE INVENTION

Many products, including consumer and professional products, are more effectively used by an end user when they include a feature that indicates a particular condition or degree of use. An example of a visual indicator is a color indicator. Color indicators can either indicate a change in condition or a degree of use through a change from “no color” to “color” (or vice versa) or through a change from one color to a different color.

Exemplary conditions that could be monitored using a color indicator include physical conditions such as the presence of moisture and chemical conditions such as a change in pH. Exemplary consumer products that could be more effective and deliver more benefits to end users by incorporating a suitable color indicator include absorbent articles, facial tissues, bath tissue, paper towels, household cleaning items and personal cleaning wipes. Exemplary professional products that could be more effective and deliver more benefits to end users by incorporating a suitable color indicator include products for medical use, safety garments, industrial cleaning products and nonwoven materials.

Color indicators are well known and are available in various forms. Desirable performance attributes include durability and good retention (i.e. the color indicator remains where intended and does not leach out into other components of the product within which it is being used). Depending on the product application, it may also be desirable to have the structure in which the color indicator is used be wettable, but water insoluble. For purposes of applying the color indicator to a component of a product, it may also be desirable to have a color indicator that can be applied in liquid form at room temperature. When the color indicator is in a liquid form at room temperature, the color indicator can be printed (just like an ink composition) onto the desired component of a product.

Examples of how color indicators are already incorporated into consumer products include diapers that have wetness sensors. Some of the wetness sensors used in diapers change color to indicate wetness while others lose color in response to wetness (i.e. the color fades or disappears when it is dissolved by water). The concept of incorporating a color-changing composition into a wearable article (such as a disposable diaper) is known in the art. For example, U.S. Pat. No. 7,159,532 issued to Klofta et al. (hereinafter “the '532 patent”) is directed to wetness indicating compositions having improved colorant retention and durability for use with wearable articles. The wetness indicating compositions of the '532 patent have a first binding agent and a second binding agent. The first binding agent immobilizes a colorant when the colorant is in its initial color state and the second binding agent immobilizes the colorant when the colorant is in its final color state. The component materials used in the examples provided in the '532 patent are solid at room temperature as indicated by the description that they need to be melted in order to combine them. While the wetness indicating compositions of the '532 patent are capable of changing color in response to a stimulus, they are not capable of being applied to an article in liquid form at room temperature.

While the color-changing compositions known in the art provide certain benefits, there remains a need for a film-forming composition that can be applied to a substrate. There also remains a need for a composition that is durable, has good retention and that shows rapid and dramatic color change when the composition is used in a product. When the purpose of the composition is to detect the presence of wetness, there remains a need for a composition that is water-resistant and water-insoluble. Further, there remains a need for a composition that is fluid and can be applied, such as by printing, at room temperature so that the composition can be applied to a substrate without heating.

### SUMMARY OF THE INVENTION

The present invention is directed to a multiple-component material that includes a film layer formed on a substrate. The film layer includes a color-changing composition that changes color when a change in physical or chemical condition is detected. The multiple-component materials of the invention may be used in personal care articles, such as the outer cover component of a disposable diaper. The color-changing composition includes a water-insoluble, film-forming polymer, a pH adjuster and a water-insoluble particle. The water-insoluble particle has a charged colorant immobilized on it. The charged colorant can be a pH indicator capable of changing color in response to the presence of wetness. When the multiple-component material is used as part of the outer cover component of a disposable diaper, the charged colorant is in contact with the absorbent core of the diaper where fluid is stored during use. The color-changing composition is fluid at room temperature and may be applied as an ink would be applied to the substrate, such as by printing or stamping. The color-changing composition may be dissolved in an organic solvent that acts as a carrier and later evaporates after the color-changing composition is applied to the film layer or forms the film layer itself.

The water-insoluble, film-forming polymer may be selected from acrylate/acrylamide copolymers, polyurethane adhesives, and copolymers of vinylpyrrolidone and dimethylaminopropyl methacrylamide. The water-insoluble, film-forming polymer is solid at room temperature, but soluble in a volatile organic solvent or an organic mixing solvent. Benefits of the color-changing composition including a film-forming polymer include that the composition adheres effectively to the substrate which prevents the composition from cracking off of the substrate when the composition is dry. When the multi-component material is used as part of the outer cover component of a disposable diaper, the film layer may have a barrier effect and may contribute to keeping fluid contained within the absorbent core of the diaper. The charged colorant may be a pH indicator where the pH indicator is selected from bromocresol green, bromophenol blue and bromochlorophenol blue. The charged colorant may be selected so that it responds by changing color to a particular physical or chemical condition. Because the charged colorant is immobilized on the water-insoluble particles, the charged colorant is stabilized and is less likely to leach away from the color-changing composition. The color-changing composition may include one or more charged colorants. When multiple charged colorants are used, the charged colorants may be selected based on the functionality that they can deliver to the color-changing composition (e.g. different color, better visibility, etc.). The color-changing composition may also include a base material. Suitable base materials include varnish bases.

Depending on the type of product that the multiple-component material is incorporated into, the film layer may be uniform across the substrate or the film layer may be formed in a pattern. If the color-changing composition is incorporated into an existing film layer, the color-changing composition may be applied uniformly or it may be applied in a pattern. Whether formed by the color-changing compositions of the inventions or whether formed by another material, the film layer may be formed on the substrate in one or more patterns selected from stripes, dots, geometric shapes, irregular shapes, alpha-numeric characters, anthropomorphic images, pictorial representation of animals, pictorial representation of inanimate objects, cartoon characters, logos and trademarks.

In another aspect, the present invention is directed to a multiple-component material that includes a substrate and a film layer on the substrate. The film layer includes a color-changing composition that includes 20% to 95% of a water-insoluble, film-forming polymer; 0.1% to 20% of a pH adjuster; and 1% to 20% of a water-insoluble particle, where the water-insoluble particle has 0.1% to 10% of a charged colorant immobilized on it. Similarly, in another aspect, the present invention is directed to a disposable absorbent article including a multiple-component material. The multiple-component material includes a substrate and a film layer on the substrate. The film layer includes a color-changing composition that includes 20% to 95% of a water-insoluble, film-forming polymer; 0.1% to 20% of a pH adjuster; and 1% to 20% of a water-insoluble particle. The water-insoluble particle has 0.1% to 10% of a charged colorant immobilized on it.

In a different aspect, the present invention is directed to a method of forming a multiple-component material. The method includes a step of mixing the components of a color-changing composition with an organic solvent to form a mixture. The color-changing composition includes a water-insoluble, film-forming polymer; a pH adjuster; and a water-insoluble particle. The water-insoluble particle has a charged colorant immobilized on it. The method also includes a step of applying the mixture to a substrate. After the applying step, the method includes a step of allowing the mixture to dry; during this step of allowing the mixture to dry, the organic solvent evaporates and the color-changing composition of the mixture forms a film on the substrate. Therefore, the presence of the organic solvent is to facilitate the step of applying the mixture to a substrate. The organic solvent does not remain with the color-changing composition after the mixture dries as a film on the substrate. The method of the invention is more efficient and lower cost than known methods because the mixture can be applied to the substrate at room temperature and without heating because the mixture is liquid at room temperature.

These aspects and additional aspects of the invention will be described in greater detail herein. Further, it is to be understood that both the foregoing general description and the following detailed description are exemplary and are intended to provide further explanation of the invention claimed.

### DETAILED DESCRIPTION OF THE INVENTION

The present disclosure of the invention will be expressed in terms of its various components, elements, constructions, configurations, arrangements and other features that may also be individually or collectively be referenced by the term, “embodiment(s)” of the invention, or other similar terms. It is contemplated that the various forms of the disclosed invention may incorporate one or more of its various features and embodiments, and that such features and embodiments may be employed in any desired, operative combination thereof.

It should also be noted that, when employed in the present disclosure, the terms “comprises”, “comprising” and other derivatives from the root term “comprise” are intended to be open-ended terms that specify the presence of any stated features, elements, integers, steps, or components, and are not intended to preclude the presence or addition of one or more other features, elements, integers, steps, components, or groups thereof.

The present invention relates to multiple-component materials that include a color-changing composition. Unlike prior art color-changing compositions, the color-changing compositions of the present invention are fluid suspensions at room temperature and can be applied to a substrate without heating. For example, the color-changing composition may be printed like an ink onto a substrate at room temperature. This feature of the color-changing compositions makes them easier to handle during the manufacture of the articles to which they are applied. Further, the color-changing compositions of the present invention form a film when they are applied, such as by printing, to a substrate and dried. A benefit of the color-changing composition forming a film on the substrate is to afford good adherence and to prevent cracking.

The color-changing compositions of the invention may include an organic solvent as a vehicle for the compositions to be applied to a substrate where the organic solvent evaporates after application. When in the form of a film layer on a substrate, the color-changing compositions of the invention are wettable but insoluble in water. This feature makes the color-changing compositions desirable for use in articles where the compositions will be exposed to wetness. The feature also results in color-changing compositions that are durable and that are resistant to leaching out of the film layer. The film layer may be formed on the substrate in a desired pattern including stripes, dots, geometric shapes and irregular shapes and combinations of such pattern elements. The film layer may also be formed on the substrate as an alpha-numeric character, an anthropomorphic image, a pictorial representation of an animal, a pictorial representation of an inanimate object, a cartoon character, a product or company logo and a trademark or brand or combinations of such pictorial elements.

The present invention is directed to a multiple-component material that includes a substrate and a film layer. The substrate may be in the form of a porous foam, a reticulated foam, cellulose tissues, a plastic film, a woven material or a nonwoven material. Suitable plastic films that may be used to form the substrate include polyethylene films and polypropylene films. Suitable woven materials include woven materials made from natural fibers, synthetic fibers or combinations of natural and synthetic fibers. Natural fibers include cotton, silk and wool fibers and synthetic fibers include polyester, polyethylene and polypropylene fibers. Suitable nonwoven materials include nonwoven materials made through traditional techniques such as spunbond, meltblown and bonded carded web materials. The spunbond, meltblown and bonded carded web materials may be made from suitable synthetic fibers such as polyester, polyethylene and polypropylene fibers. The substrate may include combinations of the materials identified above such as a substrate that includes both a porous foam and a nonwoven material or a substrate that includes both a plastic film and a nonwoven material.

The multiple-component materials of the invention also include a film layer that is adhered to the substrate. The film layer includes a color-changing composition. The color-changing composition includes a water-insoluble, film-forming polymer. The film layer may be formed by the color-changing composition itself or the color-changing composition may be applied to or incorporated into the film layer. Because the color-changing compositions of the invention are fluid suspensions at room temperature, they can be applied through printing or stamping either directly onto the substrate (thereby self-forming the film layer) or onto a pre-existing film layer associated with the substrate.

The color-changing composition includes a water-insoluble, film-forming polymer. The water-insoluble, film-forming polymer is solid at room temperature, but soluble in a volatile organic solvent or an organic mixing solvent so that the color-changing composition is liquid at room temperature. Desirably, the water-insoluble polymers/copolymers have a substantial amount, greater than about 0.5% by weight, of polar atoms such as oxygen and nitrogen. The polar atoms may be present in polar functional groups such as amides, carboxylic acids and esters. Preferably, the water-insoluble polymers/copolymers are soluble in a volatile organic solvent such as ethanol, acetone, methanol, acetonitrile, tetrahydrofuran, benzene, toluene and mixtures of such solvents. The water-insoluble, film-forming polymer and the other components of the color-changing composition can be dissolved in the organic solvent prior to application onto the substrate. When the mixture of the color-changing composition and the organic solvent is formed, the mixture is liquid at room temperature. The volatile organic solvent evaporates when the color-changing composition is either applied to the film layer or forms the film layer. Suitable water-insoluble, film-forming polymers include acrylate/acrylamide copolymers, polyurethane adhesives, copolymers of vinylpyrrolidone and copolymers of dimethyl aminopropyl methacrylamide. Commercially-available suitable polymers include DERMACRYL 79 polymer and AMPHOMER HC polymer, both of which are acrylate/octylacrylamide copolymers available from Akzo Nobel. Another example of a commercially-available suitable polymer is GANTREZ SP polymer, which is a monoalkyl ester of poly(methyl vinyl ether/maleic acid) copolymer available from International Specialty Products Inc. The color-changing compositions of the invention include a water-insoluble, film-forming polymer in an amount of from 20% to 95% of the total weight of the color-changing composition. Desirably, the color-changing compositions of the invention include a water-insoluble, film-forming polymer in an amount of from 60% to 90% of the total weight of the color-changing composition.

The color-changing composition also includes a component that forms a second, or distinct, phase to the water-insoluble, film-forming polymer. That component is a water-insoluble particle where the water-insoluble particle has a charged colorant immobilized on it. Desirably, the water-insoluble particles have a large surface area on which the charged colorant can be non-diffusively immobilized. The charged colorant may be covalently attached to or may be physically adsorbed onto the surface of the particles. The stable absorption of the charged colorant onto the water-insoluble particles increases the stability of the charged colorant and reduces leaching of the charged colorant out of the color-changing composition. Suitable water-insoluble particles include highly-charged (either positively or negatively charged) resin particles, including ion exchange resins such as REILLEX HPQ ion-exchange resin (a Poly(4-vinylpyridine), cross-linked, methyl chloride quaternary salt) commercially available from Reilly Industries, Inc. and sold through Sigma-Aldrich. Other examples of suitable water-insoluble particles include latex particles made of polystyrenes and polymethylacrylates that have surface functional groups to allow covalent attachment of the charged colorants. Additionally, inorganic particles such as clays and zeolites that allow absorption of oppositely-charged charged colorants may be used as water-insoluble particles. The water-insoluble particles may be solid or porous. Water-insoluble particles that are porous may be desirable when it is necessary to absorb/adsorb a greater quantity of charged colorant. The size of the water-insoluble particles may range from 10 nanometers to 10 micrometers and desirably from 200 nanometers to 2 micrometers. The color-changing compositions of the invention include water-insoluble particles in an amount of from 0.2% to 20% of the total weight of the color-changing composition. Desirably, the color-changing compositions of the invention include water-insoluble particles in an amount of from 2% to 10% of the total weight of the color-changing composition.

The color-changing composition of the invention also preferably includes a charged colorant that functionally acts as a pH indicator. The charged colorant is non-diffusively immobilized on the water-insoluble particle. The charged colorant is “non-diffusively immobilized” on the water-insoluble particle when the presence of the charged colorant can be reproducibly measured and when the charged colorant is present in sufficient amount to effectuate a change in color visible to the unaided human eye when the color-changing composition is exposed to a pH change (where the pH change would be sufficient to cause a change in color for the charged colorant by itself). The charged colorant may be a neutral pH indicator, a charged pH indicator or a zwitterionic pH indicator. The charged colorant may be covalently attached to the surface of the water-insoluble particles or physically absorbed through hydrophobic interactions or charge-charge interactions. When the water-insoluble particle is charged, the charged colorant is desirably oppositely charged for immobilization. When the water-insoluble particle is neutral and has surface functional groups, the charged colorant is desirably covalently immobilized.

The charged colorant desirably changes color at either a pH greater than about 9.5 or a pH lower than about 5.5. The color change may be from color to colorless, colorless to color or from one color to another color. The charged colorant has the charged functional groups either in the core chromophore structure or derivatized in pendent groups. The charged colorant may be derivatized as a polymer. Examples of suitable charged colorants include the following: gentian violet (methyl violet), leucomalachite green, methyl yellow, bromophenol blue, Congo red, methyl orange, malachite green, brillian green, crystal violet, erythrosin B, methyl green, methyl violet 2B, picric acid, napthol yellow S, quinaldine red, Eosin Y, basic fuchsin, 4-(p-anilinophenylazo)benzene-sulfonic acid, sodium salt, phloxine B, bromochlorophenol blue W.S., ethyl orange, bromocresol nile blue A, thymolphthalein, aniline blue W.S., alizarin yellow GG, morgant orange I, tropaeolin O, orange G, acid fuchsin, thiazol yellow G, indigo carmine, phenolphthalein, thymolphthalein, alizarine yellow R, bromocresol green and their respective derivatives. The color-changing compositions of the invention include a charged colorant in an amount of from 0.1% to 10% of the total weight of the color-changing composition. Desirably, the color-changing compositions of the invention include a charged colorant in an amount of from 1% to 5% of the total weight of the color-changing composition. The color-changing compositions of the invention may include more than one charged colorant. One or more charged colorants that have visually different colors may be combined or charged colorants having the same visual color may be combined.

In addition to the other components, the color-changing composition includes a pH adjuster. The pH adjuster is any molecule or composition that may be used to control the pH of the color-changing composition. The pH adjuster may be an acid, a base or a combination of both such as would be found with a buffering composition. The pH adjuster is selected in conjunction with the choice of charged colorant to be used in the color-changing composition. For example, if the color-changing composition includes a charged colorant that has a color transition point that occurs at a pH of lower than 5.5, the selected pH adjuster is desirably an acid to make the pH of the color-changing composition acidic. If the color-changing composition includes a charged colorant that transitions color at a pH higher than 9.5, the selected pH adjuster is desirably a base to make the pH of the color-changing composition basic. Examples of suitable acid pH adjusters include organic acids, inorganic acids and polymeric acids; more specifically, examples include citric acid, oxalic acid, tartaric acid, salicylic acid, palmitic acid and stearic acid. Examples of suitable base pH adjusters include organic bases, inorganic bases and polymeric bases; more specifically, examples include sodium hydroxide, sodium carbonate, sodium bicarbonate, sodium borate, potassium hydroxide, polymeric amines, dendrimeric amine and 1,3-pentanediamine. Combination pH adjusters that have a buffering effect include acetic buffer, borate buffer and carbonate buffer. Desirably, the pH of the combination pH adjuster is either greater than 10 or lower than 5. Typically, the combination pH adjuster is in solution form and the concentration of the buffer may range from about 0.01 milliMolar to about 1000 milliMolar and desirably range from about 1 milliMolar to about 20 milliMolar, depending on the combination pH adjuster selected. The color-changing compositions of the invention include a pH adjuster in an amount of from 0.1% to 20% of the total weight of the color-changing composition. Desirably, the color-changing compositions of the invention include a pH adjuster in an amount of from 0.5% to 5% of the total weight of the color-changing composition.

Benefits of the pH adjuster include stabilizing the charged colorant against premature color changes that may be caused by exposure to humid environments. For example, the pH adjuster is believed to maintain a stable pH, such as a low pH environment with an acidic pH adjuster, around the charged colorant even when the film layer is exposed to high humidities.

The color-changing composition of the invention may also include a base material. The base material may be a small molecule, a polymeric material or a mixture of small molecules and polymers. Examples of suitable small molecule base materials include glycols, including triglycerols and their derivatives. Examples of suitable polymeric materials that may be used as base materials include polyvinyl alcohol, polyvinyl pyrrolidone, polyethylene glycol, water-soluble derivatives of polyacrylates and polyacrylamides, poly(hydroxyethyl methacrylates), poly(hydroxylethyl acrylates), carboxymethyl cellulose, gelatin and gum Arabic. Another suitable base material for the color-changing compositions of the invention is a varnish base such as a nitrocellulose compound based varnish or a phenolic-modified co-solvent-type polyamide resin-based varnish. The color-changing compositions of the invention may include a base material in an amount of from 20% to 95% of the total weight of the color-changing composition. It is believed that the base material may help the stability of the color-changing composition, such as by increasing the stability of the suspension of the water-insoluble particles. It is also believed that the base material may improve the adhesion of the color-changing composition to the substrate of the multiple-component material. The base material may be water-soluble or water-insoluble while the charged particles are water-insoluble.

When the components of the color-changing composition are formed as a film layer on the substrate, they must first be dissolved or suspended in an organic solvent that later evaporates. The result of the color-changing composition forming a film layer on the substrate is the multiple-component material of the invention. The organic solvent may be a single solvent or a mixture of solvents. Suitable solvents to carry the color-changing composition include ethanol, isopropanol, acetone, methanol, acetonitrile, tetrahydrofuran, benzene and toluene. An example of a useful application of the present invention is to apply the color-changing composition to a nonwoven substrate that is used as a component of a disposable absorbent article. More specifically, the nonwoven substrate may be a spunbond or other nonwoven material that is used to form the outer cover of a disposable diaper. The substrate may also be a polyethylene film that is adhered to a nonwoven material to form the outer cover of a disposable diaper. The film layer of the present invention may be formed on a nonwoven substrate or on another film. Because the color-changing compositions of the present invention are fluid at room temperature, they can be easily applied through printing to a substrate. Because the outer cover of an absorbent article, such as a disposable diaper, is typically adjacent the absorbent structure of the article, the color-changing composition is applied to a component that is in proximity to the absorbent structure when the finished product is in use. Therefore, the color-changing composition can be used to indicate a change in condition of the absorbent structure, such as wetness.

An example of a color-changing composition of the invention is a composition that includes an acrylate and octylacrylamide copolymer system as the water-insoluble, film-forming polymer; this polymer can assist adhesion of the charged colorant to the substrate and provide wettability with water resistance. Another example of the color-changing composition of the invention is a composition that includes a polyurethane adhesive with amide functionality as the water-insoluble, film-forming polymer. This water-insoluble, film-forming polymer can be dissolved in an alcohol for application to the substrate (with the alcohol subsequently evaporating). The color-changing compositions of the invention are water-wettable to allow rapid color change of the charged colorant component, but are water-insoluble to prevent leaching of the charged colorant. These are desirable performance attributes. In order to prepare the color-changing compositions of the invention, the water-insoluble particle is mixed with an oppositely-charged charged colorant and a pH adjuster.

The following are various examples that illustrate aspects of the present invention:

### EXAMPLE 1

Preparation of Exemplary Water-Insoluble Particle #1:

As a first step, 100 mg citric acid (acid pH adjuster) and 10 mg Bromocresol green (charged colorant) were dissolved and mixed in 100 ml of water to make a dye solution. Three grams (3 g) of an oppositely-charged water-insoluble particle, REILLEX HPQ ion exchange resin (a cross-linked positively charged polyvinyl pyridine, commercially available from Sigma Aldrich), was immersed in 20 ml of the dye solution. The purpose of immersing the water-insoluble particles in the dye solution is to allow the pH adjuster and the charged colorant to adsorb onto the water-insoluble particles. Initially, the water-insoluble particles were colored greenish blue. After adding 700 mg of excess citric acid, the color of the particles changed to yellowish-green. The dye solution was then decanted off of the water-insoluble particles. The colored water-insoluble particles were dried in air and when dried, had a white-yellow appearance. The colored water-insoluble particles were ground into fine powder having an average size of 715 nm, characterized by dynamic light scattering (DLS) in ethanol. The ground powder was used in examples #2 and #3 below.

### EXAMPLE 2

Preparation of Exemplary Multiple Component Material #1:

In this example, DERMACRYL 79 polymer is used as the water-insoluble, film-forming polymer. The DERMACRYL 79 polymer is diluted in ethanol to a 10% (by weight) solution. The ethanol is the organic solvent that later evaporates when the multiple component material is formed. In order to form the color-changing composition, 0.1 grams of the water-insoluble particles of Example 1 are mixed with 0.5 milliliters of the diluted DERMACRYL 79 polymer solution. This color-changing composition was applied to outer cover film from a HUGGIES diaper sold by Kimberly-Clark Global Sales, LLC. The outer cover film is a polyethylene film. The color-changing composition was brushed onto the film to form a multiple component material of the invention. After one hour of drying, the color-changing composition appeared as a white-yellow color on the outer cover film. The color-changing ability of the multiple component material was tested by adding a sample of water and separately, a sample of synthetic urine to the area of the outer cover film onto which the color-changing composition had been brushed. Within 15 seconds, the color of the color-changing composition changed to green. After the multiple component material was allowed to dry, the green color remained and appeared slightly-yellowish green. When another application of water was made, the color of the color-changing composition changed to blue.

### EXAMPLE 3

Preparation of Exemplary Multiple Component Material #2:

In this example, polyurethane polymer solution is used as the water-insoluble, film-forming polymer. The polyurethane polymer solution is 70% solids in methanol where the solids are 1,1,1-trimethanol propane and toluene 2,4 diisocyanate; this polyurethane polymer solution is available from Sung-Do Chemical Company in South Korea. The polyurethane polymer solution is diluted in ethanol to a 7% total solids solution. The ethanol is the organic solvent that later evaporates when the multiple component material is formed. In order to form the color-changing composition, 0.1 grams of the water-insoluble particles of Example 1 are mixed with 0.3 milliliters of the diluted polyurethane polymer solution. This color-changing composition was applied to outer cover film from a HUGGIES diaper sold by Kimberly-Clark Global Sales, LLC. The outer cover film is a polyethylene film. The color-changing composition was brushed onto the film to form a multiple component material of the invention. After one hour of drying, the color-changing composition appeared as a white-yellow color on the outer cover film. The color-changing ability of the multiple component material was tested by adding a sample of water and separately, a sample of synthetic urine to the area of the outer cover film onto which the color-changing composition had been brushed. Within 5 minutes, the color of the color-changing composition changed to green. After two hours of exposure to water, the color of the color-changing composition changed to blue.

### EXAMPLE 4

Preparation of Exemplary Water-Insoluble Particle #2:

Six grams (6 g) of an oppositely-charged water-insoluble particle, REILLEX HPQ ion exchange resin (a cross-linked positively charged polyvinyl pyridine, commercially available from Sigma Aldrich), was immersed in 9 ml of water solution with 100 mg Bromocresol green (charged colorant) added. The solution was stirred for five hours. After the five hours, the water-insoluble particles were filtered from the solution and washed with excess ethanol (7 milliliters×5) and excess water (7 milliliters×6) after which the particles were allowed to dry in air. The dried water-insoluble particles were then suspended in 7 milliliters of water solution to which 5 grams of citric acid (acid pH adjuster) had been added. The solution was stirred for 24 hours to keep the particles suspended and to allow for full adsorption of the citric acid into the particles. The particles were then filtered without washing and then allowed to dry in air. The dried, yellow-colored particles were then ground into a fine powder such that the ground particles had an average size of 1012 nm as characterized by dynamic light scattering (DLS) in ethanol using ELS. The ground powder was used in examples #5 and #6 below.

### EXAMPLE 5

Preparation of Exemplary Multiple Component Material #3:

In this example, DERMACRYL 79 polymer is used as the water-insoluble, film-forming polymer. The 200 mg DERMACRYL 79 polymer was dissolved in 2 ml of iso-propanol. The iso-propanol is the organic solvent that later evaporates when the multiple component material is formed. In order to form the color-changing composition, 0.2 grams of the water-insoluble particles of Example 4 are mixed with the diluted DERMACRYL 79 polymer solution. This color-changing composition (yellow in color) was applied to outer cover film from a HUGGIES diaper sold by Kimberly-Clark Global Sales, LLC. The outer cover film is a polyethylene film. The color-changing composition was brushed onto the film to form a multiple component material of the invention. After one hour of drying, the color-changing composition appeared a little greenish-yellow in color on the outer cover film. The color-changing ability of the multiple component material was tested by adding a sample of water and separately, a sample of synthetic urine to the area of the outer cover film onto which the color-changing composition had been brushed. Within 15 seconds, the color of the color-changing composition changed to green. After the multiple component material was allowed to dry, the green color remained.

### EXAMPLE 6

Preparation of Exemplary Multiple Component Material #4:

In this example, poly(vinyl acetate-co-butyl maleate-co-isobornyl acrylate) polymer as a 50% solution in ethanol was used as the water-insoluble, film-forming polymer. The ethanol is the organic solvent that later evaporates when the multiple component material is formed. The polymer solution is available from Sigma-Aldrich Chemical. 0.4 ml of the polymer solution was diluted with 1.6 ml of iso-propanol. In order to form the color-changing composition, 0.2 grams of the water-insoluble particles of Example 4 were mixed with the 2 ml of the diluted polymer solution. This color-changing composition was applied to outer cover film from a HUGGIES diaper sold by Kimberly-Clark Global Sales, LLC. The outer cover film is a polyethylene film. The color-changing composition was brushed onto the film to form a multiple component material of the invention. After one hour of drying, the color-changing composition appeared as a yellow color on the outer cover film. The color-changing ability of the multiple component material was tested by adding a sample of water and separately, a sample of synthetic urine to the area of the outer cover film onto which the color-changing composition had been brushed. Within 15 seconds, the color of the color-changing composition changed to green. After the multiple component material was allowed to dry, the green color remained as with Example 5.

While the multiple-component materials of the invention have been described in detail with respect to specific aspects thereof, it will be appreciated that those skilled in the art, upon attaining an understanding of the foregoing, may readily conceive of alterations to, variations of and equivalents to these materials. Accordingly, the scope of the present invention should be assessed as that of the appended claims and any equivalents thereto.

We claim:

1. A multiple-component material, the material comprising:
a substrate and a film layer on the substrate, wherein the film layer includes a color-changing composition, the color-changing composition comprising:
a water-insoluble, film-forming polymer;
a pH adjuster; and
a water-insoluble particle, wherein the water-insoluble particle has a charged colorant immobilized on it.

2. The multiple-component material of claim 1, wherein the water-insoluble particle has a particle size of 10 nanometers to 10 micrometers.

3. The multiple-component material of claim 1, wherein the charged colorant is covalently attached to the water-insoluble particle.

4. The multiple-component material of claim 1, wherein the charged colorant is physically absorbed onto the water-insoluble particle.

5. The multiple-component material of claim 1, wherein the water-insoluble, film-forming polymer has greater than about 0.5% by weight of polar atoms.

6. The multiple-component material of claim 1, wherein the water-insoluble, film-forming polymer is selected from acrylate/acrylamide copolymers, polyurethane adhesives, and copolymers of vinylpyrrolidone and dimethylaminopropyl methacrylamide.

7. The multiple-component material of claim 1, wherein the charged colorant is a pH indicator.

8. The multiple-component material of claim 8, wherein the pH indicator is selected from bromocresol green, bromophenol blue and bromochlorophenol blue, methyl orange, tetrabromophenol blue, ethyl orange, Congo red, methyl red and allure red.

9. The multiple-component material of claim 1, wherein the color-changing composition includes more than one charged colorant.

10. The multiple-component material of claim 1, wherein the substrate is a non-woven material.

11. The multiple-component material of claim 1, wherein the substrate is a polyolefin film.

12. The multiple-component material of claim 1, wherein the substrate is selected from a polyethylene film and a polypropylene film.

13. The multiple-component material of claim 1, wherein the color-changing composition further comprises a base material.

14. The multiple-component material of claim 13, wherein the base material is a varnish base.

15. A disposable absorbent article including the multiple-component material of claim 1 as part of an outer cover.

16. The multiple-component material of claim 1, wherein the film layer is formed on the substrate in one or more patterns selected from stripes, dots, geometric shapes, irregular shapes, alpha-numeric characters, anthropomorphic images, pictorial representation of animals, pictorial representation of inanimate objects, cartoon characters, logos and trademarks.

17. A multiple-component material, the material comprising:
a substrate and a film layer on the substrate, wherein the film layer includes a color-changing composition, the color-changing composition comprising:
20% to 95% of a water-insoluble, film-forming polymer;
0.1% to 20% of a pH adjuster; and
0.2% to 20% of a water-insoluble particle, wherein the water-insoluble particle has 0.1% to 10% of a charged colorant immobilized on it.

18. A disposable absorbent article including a multiple-component material, the material comprising: a substrate and a film layer on the substrate, wherein the film layer includes a color-changing composition, the color-changing composition comprising:
20% to 95% of a water-insoluble, film-forming polymer;
0.1% to 20% of a pH adjuster; and
0.2% to 20% of a water-insoluble particle, wherein the water-insoluble particle has 0.1% to 10% of a charged colorant immobilized on it.

19. A method of forming a multiple-component material, the method comprising the steps of:
mixing components of a color-changing composition with an organic solvent to form a mixture, wherein the components of the color-changing composition include a water-insoluble, film-forming polymer; a pH adjuster; and a water-insoluble particle, wherein the water-insoluble particle has a charged colorant immobilized on it;
applying the mixture to a substrate; and
allowing the mixture to dry, wherein during this step the organic solvent evaporates and the color-changing composition of the mixture forms a film on the substrate.

20. The method of forming a multiple-component material of claim 19, wherein the mixture is liquid at room temperature.
